(19) United States
(12) Patent Application Publication
(10) Pub. No.: US 2006/0079903 A1
(43) Pub. Date: Apr. 13, 2006

(54) Minimally invasive pedicle screw and guide support

(76) Inventor: David A. Wong, Golden, CO (US)

Correspondence Address: DORR, CARSON & BIRNEY, P.C.;ONE CHERRY CENTER, 501 SOUTH CHERRY STREET, SUITE 800, DENVER, CO, 80246, US

(21) Appl. No.: 11/221,633

(22) Filed: Sep. 8, 2005

Related U.S. Application Data
(63) Continuation of application No. 60/617,109, filed on Oct. 8, 2004.

(51) Int. Cl. A61B 17/58

(52) U.S. Cl. 606/72

(57) ABSTRACT

A minimally invasive orthopedic bone attachment system comprising a pedicle screw and guide support sleeve for insertion of the screw. The pedicle screw is a self-boring, self-tapping integral screw having a distal sharply pointed end for guiding the insertion of the screw as well as forming a borehole for the self-tapping threads of the screw. The proximal end of the screw provides a cylindrical extension which can include a recess for receiving a rotational drive tool or wrench. The length of the threads and the type of threads provided in the screw are designed for the type of orthopedic surgery that is intended. A relatively thin hollow support sleeve including an internal threaded section in the distal end is provided to match the threads and the length of the threaded portion of the pedicle screw. The proximal end of the sleeve includes a central passageway having an internal diameter that can receive the extension portion of the screw and also guide and receive a drive device for the screw. A retainer clip associated with circumferential slots spacedly indexed along the longitudinal surface of the drive device can be used to limit the depth of the screw upon insertion as well as to lock the drive device as an assembly with the screw and sleeve to form the attachment system. A small diameter passageway can be provided along the longitudinal axis of the assembly extending from the proximal end of the drive device through the screw to exit through the pointed distal end of the screw. A thin rigid rod having a point at the distal end is slidably positioned within the narrow passageway. The proximal end of the road includes a cap. A flange surface on the under portion of the cap limits the travel of the rod through the assembly. The length of the rod is determined so that it equals the length of the assembly plus an additional dimension corresponding to the anticipated installed depth of the screw. Prior to insertion of the screw, the rod is driven into the bone through the assembly. The location of the rod is ascertained by an image guidance system to determine the correct final projected location of the screw upon installation.

### RELATED APPLICATIONS

This application claims the benefit of U.S. Provisional Patent Application Ser. No. 60/617,109 filed Oct. 8, 2004.

### BACKGROUND OF THE INVENTION

1. Field of the Invention

This invention is directed to an improved pedicle screw and a tubular installation support for spinal stabilization surgery. It is more specifically directed to a threaded screw having a smooth cylindrical barrel at the proximal end and a smooth, narrow pointed distal end. A hollow cylindrical sleeve having interior threads is mated with the screw to support the screw and hold it in alignment during installation.

2. Discussion of the Background

Over the years a number of various types of threaded fasteners have been used in orthopedic surgery to hold bone fragments as well as to reattach ligaments and soft tissue to a bone section. As a result, many innovations have been provided to assist or aid in the installation of the screws into the bone as well as providing various tools and accessories for accomplishing this task.

This was especially true when it came to surgery on the spine where it was found to be advantageous to install attachment devices in the associated vertebrae to hold the vertebrae in relative position with respect to each other to allow a crack or fracture to mend or a surgical fusion to heal in a relatively short period of time. For many years the usual treatment was to place the patient's spine in traction which required the immobilization of the patient during the healing process. During this time period the patient had to be rotated for physiological reasons from front-to-back and back-to-front in order to minimize ancillary complications and problems. This process in turn had an inherent risk of re-fracturing the spinal fusion potentially prolonging the healing time for the patient.

To better understand the more recent procedures which have taken place with respect to spinal surgery and stabilization of the spine, it is necessary to note that the “pedicle” is the basal part of each side of the neural arch of a vertebrae. It represents the strong bridge connecting the anterior and posterior portions of a spinal vertebrae. Improvements to the procedure for treating patient injuries or degenerative problems in the back or spine relate to various damage such as a fracture of the vertebrae or damage to spinal discs positioned between the vertebrae. Instead of providing traction as was done in the past, recent prior art methods have been proposed for placing anchors in the pedicle portions of the vertebrae and then provide connecting instrumentation to immobilize or stabilize that affected portion of the spine to allow the bones to fuse and heal. One way of accomplishing this was to provide a metal plate which included a plurality of strategically located holes through which screws were fastened to the pedicle of the vertebrae to immobilize that portion of the spine. Other types of instrumentation to provide the fixation and immobilization of the spine have also been tried.

One of the major problems in immobilizing the spine to facilitate the healing process has been the complicated procedure for insertion and anchoring of the support screws in the pedicle. In the past, the “stacking on” approach had been utilized for the purpose of installing the pedicle screws via a large open surgical incision. This methodology had many inherent problems since it had a number of intricate steps and the need for accurate placement of each element during these steps increased the operating time and x-ray exposure for both the surgeon as well as the patient. The placement of each element was critical to the success of the healing process. Each step of the insertion of metallic elements requires the verification of placement by a conventional image guidance system, such as an x-ray fluoroscopic inspection. At times, exit penetration of the vertebrae by an element into the abdomen or nerve canal of the patient produced catastrophic results.

The “stacking on” surgical approach to spinal operations was preceded by a surgical incision located centrally along the posterior spine. This type of incision caused considerable problems from the standpoint that the length of the incision is relatively long in order to allow access by the surgeon into the required vertebrae area. This required retraction of the muscles and soft tissue to provide this access. This standard incision resulted in extensive time required for patient healing as well as problems encountered with scar tissue. The “stacking on” process begins with the insertion of a thin metal guide wire into the pedicle and adjacent vertebral body with fluoroscopic x-ray control. Progressively larger working cannulated tubes or drills are sequentially placed over the guide wire with an ultimate diameter sufficient to insert taps and anchor screws to perform a pedicle screw instrumentation. X-ray verification is required at each step.

The first or initial element used is the guide wire which must be quite thin. The guide wire is sometimes difficult to place and maneuver, and especially if the patient is large and has a corresponding mass of soft tissue tending to push into the incision. Next a cannulated working tube or drill is positioned over the guide wire and into the incision to effectuate the next step. A bent guide wire can bind inside the tube or drill in the “stacking on” procedure. As the working tube is inserted over the guide wire, the guide wire has been noted to occasionally push forward through the vertebrae and actually penetrate the abdomen. The guide wire can also bind within the tube and can also come out of the track when the exchange of tubes or instruments occurs. When this happens the pathway is lost and a new guide wire penetration must be accomplished to re-establish the alignment of the track. The original incisions were large in order to allow the surgeon to visually guide and position the “stacking on” process for the insertion of traditionally designed pedicle screws.

It became readily apparent that large incisions were counterproductive to the success of the spinal operation and the healing of the patient. To accelerate the healing of the incision, the incisions became smaller and in some cases were placed several inches on each side of the central portion of the spine to accommodate the surgical procedures. This arrangement further complicated the positioning and insertion of the stabilization instrumentation. Fluoroscopic x-ray position verification of the instrumentation elements became even more critical. The reduction in the incision size and the subsequent reduced damage to the muscle and the soft tissue helped to improve the post-operative condition of the patient but made the surgical procedure even more technically difficult and increased x-ray exposure.

It became readily apparent to the applicant that an even more minimally invasive procedure for the insertion of the anchor screws for stabilization instrumentation of the lumbar area of the spine would greatly improve the post-operative condition of the patient. For this reason, a pedicle screw insertion which will eliminate the “stacking on” prior art process would be of considerable benefit. In addition, it was realized that a cannulated-type screw or anchoring element was no longer required if the guide wire could be eliminated. Also it was found that it would be highly desirable to include a sleeve to support and hold or retain a self-contained integrally formed pedicle screw that could be inserted directly through a much smaller incision in the skin. The sleeve would further serve to protect the soft tissues from damage by the threaded portion of the pedicle screw.

This movement towards more minimally invasive spinal surgery has compelled a new look at the design of instruments and implants which were originally manufactured for open stabilization and fusion of the spine. Instrumentation appropriate for situations of open surgery with direct visualization of anatomical landmarks did not necessarily convert for use in minimally invasive solutions.

These circumstances have led to a radical shift in the approach to instruments and implants now proposed for improved spinal surgery. Rather than using a “stacking on” approach the applicant uses an instrument working sleeve in conjunction with a pedicle screw which is assembled outside the body to provide a single combined integral device incorporating the functions of the guide wire, installation sleeve and pedicle screw implant. This improved apparatus allows a single step establishment of the orientation to the insertion site for the screw and facilitates an additional reaming action for establishing the appropriate track for the screw within the pedicle.

The single integrated assembly approach allows for a “reverse stacking on” process. Once the pedicle screw has been inserted to the appropriate depth, the working or installation sleeve can be easily withdrawn in a single step. The threaded portion of the sleeve provides a firm fixation for the screw as it is installed with the release of the sleeve as the last threads of the screw exit the sleeve. The reverse is also true for the extraction of the screw when the time becomes appropriate.

The single assembly approach also allows the use of a more substantial sized guide portion and removes the concerns about complications of the guide wire bending during use. In addition, the novel threaded design of the new installation support sleeve is a departure from the traditional smooth bored sleeves which act as a working portal only. Thus, the threaded nature of the support sleeve helps provide security and stability for single step guidance, insertion and fixation. The support sleeve also can incorporate a guide function for the insertion and rotation of the driver.

This new improved method requires a significant design change for the pedicle screws as well. The new pedicle screw incorporates a solid guide distal portion of the pedicle screw with a sharp cutting tip and a smooth shaft. The length of the guide portion of the screw is of sufficient length to allow penetration of the lumbar pedicle (approximately 8-10 millimeters). Percutaneous insertion of the screw implant to the depth of the guide portion allows a one step definition of the appropriate track for the pedicle screw. A smooth surfaced cylindrical barrel or stem on the proximal end of the screw provides for attachment of appropriate instrumentation. A low height on this stem portion results in less soft tissue irritation and damage with the use of spine fixation instrumentation.

Information Disclosure Statement. This section complies with the applicant's requirement to disclose all of the prior art of which he is aware and which may apply to the examination of the present application.

The Stednitz et al. patent (U.S. Pat. No. 5,098,435) discloses a bone stabilizing system which includes a fixation device which comprises a metal cannula defined by a hollow cylindrical shaft having drilling teeth at one end and a receiving device at the other end and a plurality of threads therebetween. A solid non-cannulated embodiment of the fixation device is also shown. Intersecting a portion of the threads is at least one flute which is defined by two substantially orthogonal surfaces and an elongated slot which penetrates the wall of the cannula so as to provide fluid communication.

The Konieczynski patent (U.S. Pat. No. 6,183,478) discloses a device for affixing a bone plate to spinal bone which includes a sleeve, bias member and a fixation member. The fixation member is a screw-type device. The elongated hollow sleeve does not have any internal threads and the inner diameter is greater than the fixation device. The fixation member is housed within the sleeve and during installation is pushed out through the distal end of the sleeve in order to contact and engage the bone through the bone plate.

The Walawalkar patent (U.S. Pat. No. 5,904,685) discloses an apparatus for inserting a screw into a tunnel in a surgical site. The apparatus includes a screw which is inserted in a cannulated sheath or sleeve for guiding the insertion of the screw. A protrusion on a cantilevered arm formed in the wall of the sheath temporarily holds the screw in position at the distal end of the sheath during insertion. This protrusion is relatively flexible and merely holds the screw in place within the sheath. It does not stabilize or aid the support or alignment of the screw during the insertion process.

The Coleman patent (U.S. Pat. No. 5,645,547) also shows a cannulated screw which is inserted or installed onto a keyed rotatable insertion shaft. The shaft has a point on the end which is used to provide a guide tunnel for insertion of the screw.

The Fucci patent (U.S. Pat. No. 5,607,432) discloses a retriever for removing a threaded bone anchor from an implantation site. The retriever comprises an elongated shaft having an anchor engaging means at its distal tip for engaging the drive portion of the threaded bone anchor and for turning it in a direction to remove it from the implantation site. A concentric anchor engaging sleeve is adapted to move longitudinally relative to the elongated shaft in order to engage the threaded body of the anchor as it is removed from the implantation site. The sleeve has one or two internal threads which engage the anchor upon its retrieval so that it will be retained within the sleeve so that the anchor can be removed from the implantation site.

The Simon et al. patent (U.S. Pat. No. 6,415,693) and Leibinger et al. patent (U.S. Pat. No. 4,763,548) show sleeve-type screwdrivers which are used to retain fixation devices such as screws during orthopedic surgery. Both of these devices comprise a gripping sleeve at the free end of the device which has a radially expandable portion for receiving and holding the screw. The expandable or gripping portion in both patents is effective by sliding a sleeve longitudinally with respect to the gripping or expandable portion. A handle is axially movable to engage the screw or fixation device so that it can be rotated for insertion into the bone. Neither of these patents disclose the use of full length threads provided internally within the sleeve for retention of the screw or fixation device during the installation process.

### SUMMARY OF THE INVENTION

The present invention is directed to a minimally invasive pedicle screw and guide support sleeve for insertion of the screw. The screw in this context is intended as an anchor for support of instrumentation to stabilize and support vertebrae in the spinal column to allow fusion and healing within the spine. It is to be understood that although the discussion herein is directed to surgical procedures with the spine it is also possible that the anchor screw and guide support described herein can be used in various types of orthopedic surgery dealing with anchoring and fusion of the bone as well as the attachment of ligaments and tendons to the bone.

This system is composed of a novel self-boring, self-tapping integral screw having a distal sharply pointed end for guiding the insertion of the screw as well as forming the preliminary borehole for the self-tapping threads of the screw.

The proximal end of the screw provides a smooth cylindrical barrel which can include a recess in the end for receiving a rotational drive tool or wrench. The body of the screw incorporates an elongated portion which is suitably threaded to allow the screw to be inserted into the pedicle portion of the vertebrae and rigidly anchored. The first several threads are self-tapping threads provided on the distal end of the threaded portion of the body which allows the threads as they are turned to cut into the bone matter and self-tap the inner surface of the borehole formed by the guide portion of the screw.

One or more longitudinal flutes can be provided partial or full length along the threaded body portion of the screw to allow relief of the bone matter cut by the self-tapping threads as the screw is inserted into the bone.

A relatively thin hollow support sleeve or tube is provided which includes an internally threaded section which matches the threads and the length of the threaded portion of the pedicle screw. The distal end of the sleeve has a rounded or beveled edge so as to minimize soft tissue injury or damage that is caused by the insertion of the pedicle screw and the sleeve percutaneously during the installation process.

The proximal end of the sleeve has an internal diameter that can match the diameter of the barrel or stem portion of the screw or can be substantially larger than this diameter to allow the insertion of a drive tool which can have a hexagonal coupler in its end which will mate with a hexagonal drive portion provided at the proximal end of the threaded portion of the screw. It is readily apparent that any type of suitable drive coupler or connection can be used for engagement of the drive tool with the pedicle screw as desired. The internal diameter of the proximal end of the support sleeve can be varied to accommodate different types of drive devices.

The pedicle screw as described herein is intended to provide an anchor for instrumentation that can be used to interconnect the stem portion of a plurality of pedicle screws provided in the vertebrae of a patient's spine. The instrumentation can include rods having various lengths which are attached to the stem portion of the screws for stabilizing and fixating the vertebrae of the spine of the patient which will allow the spine to quickly heal without the tissue trauma normally encountered with extensive percutaneous spinal surgery. The use of the support sleeve in conjunction with the installation of the pedicle screw is critical in that the screw is firmly held within the sleeve as the sleeve is inserted through a small incision in the skin over the pedicle portion of the vertebrae so that the soft tissue and muscle is not further damaged by the threads of the screw during rotation and insertion of the pedicle screw and the screw remains properly aligned.

The support sleeve can be reused for the insertion and withdrawal of any number of pedicle screws during the surgical process which minimizes the cost of this procedure as well as providing minimally invasive insertion of the pedicle anchor screws required for the stabilization and healing of the patient's spine.

The support sleeve in conjunction with the shaft of the drive tool used for driving the screw can incorporate indices or marks on the shaft of the drive tool which correspond with the threaded length of the pedicle screw during the insertion process. The indices can also take the form of peripheral slots formed in the shaft of the tool at various predetermined locations along the shaft which identify the depth of the pedicle screw during the installation. A retainer clip or locking ring having an extended handle can be inserted into the peripheral slots on the shaft of the tool with one coinciding with the proximal end of the support sleeve. In another arrangement, slots can be provided through opposite sides of the support sleeve which align with a circumferential slot on the driver tool so that the retainer clip will temporarily lock the drive tool and the pedicle screw with respect to the support sleeve. This locking process provides a rigid assembly prior to the installation and insertion of the screw with the retainer clip repositioned to the proper slot on the shaft of the drive tool upon the start of the insertion process. In this way the retainer clip will limit the insertion depth of the pedicle screw to prevent the screw from being inserted to a position where it could exit the pedicle portion of the vertebrae and enter the abdominal cavity of the patient. Other indices or marks can be provided strategically along the shaft of the drive tool or the sleeve to provide indication of various depths of the screw as it is inserted.

In another embodiment of the invention, a locator rod can be incorporated into the assembly so as to project or predict the location and depth of the orthopedic screw prior to its installation. In most cases, the screw and driver device are aligned along a single longitudinal axis. A thin central passageway can be formed to coincide with the longitudinal axis of the assembled screw and driver. A thin rod is then slidably positioned within the central passageway. The rod includes a sharp point at the distal end and an enlarged knob or stop at the opposite proximal end. The length of the rod is predetermined as the combined length of the screw and driver plus the length of the threaded portion of the screw.

With the assembly properly positioned and aligned with respect to the bone mass, the rod is tapped with a small instrument to force the rod into the bone until the rod stop contacts the end of the driver. An image guidance system can be used to verify the location and depth of the distal end of the locator rod with minimum exposure to the patient and surgeon. The locator rod then projects the future position of the screw prior to its actual installation. If desired, spacer disks can be installed over the rod to limit the penetration depth of the locator rod if only a partial thread depth penetration of the screw is intended.

The present invention has been briefly described herein but it is understood that other aspect and features of the invention may become apparent from the following detailed description of the invention when it is considered in conjunction with the accompanying drawings.

### BRIEF DESCRIPTION OF THE DRAWINGS

FIG. 1 is a pictorial view showing an x-ray image of the spine of a patient with the insertion of a plurality of pedicle screws according to the present invention;

FIG. 2 is a side partially sectioned view of the lumbar area of a patient's spine showing the insertion of a plurality of pedicle screws;

FIG. 3 is an enlarged pictorial view of the pedicle screws displayed in FIG. 2 along lines 3-3;

FIG. 4 is a side pictorial view of the pedicle screws inserted in the vertebrae of the patient's spine as shown in lines 44 of FIG. 1;

FIG. 5 is a cross-section of a vertebrae of the patient's spine showing the placement of the pedicle screws according to lines 5-5 of FIG. 4;

FIG. 6 is an assembly view of the pedicle screw insertion system of the present invention;

FIG. 7 is a partially assembled view of FIG. 6 showing the pedicle screw threaded into the support sleeve;

FIG. 8 is the assembled view of the pedicle screw insertion system according to the present invention;

FIG. 9 shows a cross-section view of the assembled pedicle screw taken along lines 9-9 of FIG. 8;

FIG. 10 is a cross-section view taken along lines 10-10 of FIG. 8;

FIG. 11 is a cross-section view taken along lines 11-11 of FIG. 8;

FIG. 12 is a cross-section view taken along lines 12-12 of FIG. 8;

FIGS. 13-15 are pictorial views showing the insertion of the pedicle screw into the vertebrae of a spinal column;

FIG. 16 is a perspective view of a pedicle screw insertion system showing a transverse handle provided on the support sleeve;

FIG. 17 is a perspective view of the system disassembled showing a hexagonal ring on the screw above the threaded portion with a hexagonal socket provided on the drive tool;

FIG. 18 is a sectional view showing the hexagonal receptacle on the drive tool engaging the hexagonal ring on the pedicle screw with the drive tool also engaging a receptacle in the proximal end of the screw;

FIG. 19 is a perspective view showing indicia slots provided on the shaft of the drive tool with a retainer clip installed;

FIG. 20 is a perspective view of the retainer clip shown in FIG. 19;

FIG. 21 is a partial sectional view showing the drive tool and the retainer clip inserted into the support sleeve with the pedicle screw partially extended;

FIG. 22 is the assembled view of the pedicle screw insertion system which includes the locator rod embodiment;

FIG. 23 is the assembled view as shown in FIG. 22 wherein the locator rod extends beyond the proximal guide end of the pedicle screw to provide the projected location of the installed screw;

FIG. 24 is a cross section taken along lines 24-24 of FIG. 23;

FIG. 25 is a side cross sectional view of the assembled pedicle screw insertion system showing the locator rod; and

FIG. 26 is an enlarged partial sectional view showing the rod and its depth controlling cap.

### DETAILED DESCRIPTION OF THE INVENTION

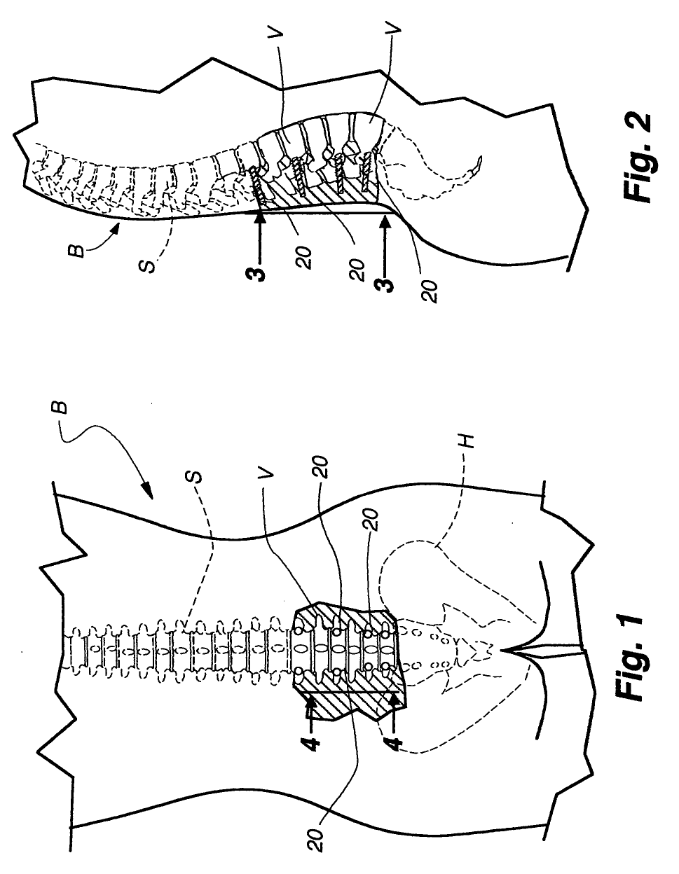

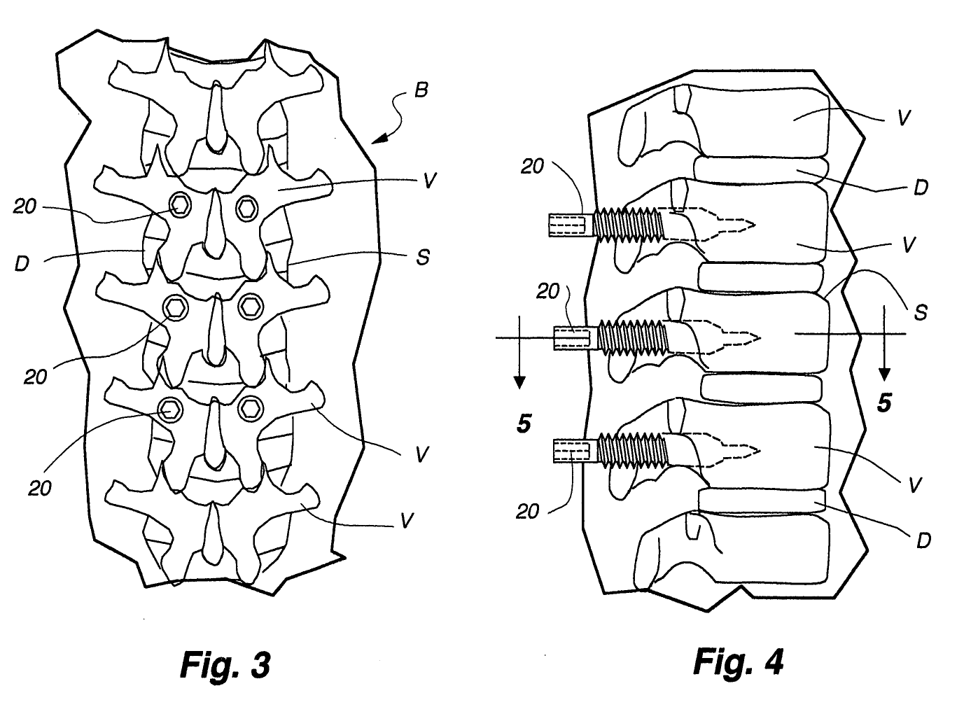

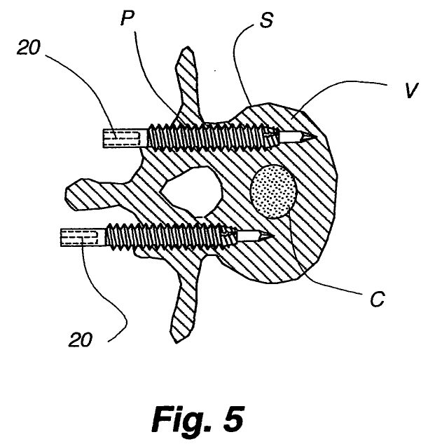

Turning now more specifically to the drawings, FIGS. 1-5 show a partial pictorial view of a patient's body B revealing the position of the spinal column S, the pelvis bone H as well as the individual vertebrae V which makes up a portion of the spine. Intervertebral discs D which are positioned between each of the vertebrae V go together to protect and support the spinal cord and nerves C positioned within the structure making up the spinal column S.

In each of the views is seen a pedicle or orthopedic screw 20 which has been strategically positioned and installed within the pedicle portion of certain vertebrae V. The primary purpose of the pedicle screw 20 is to provide a rigid anchor in the affected and adjacent vertebrae so that the vertebrae can be rigidly supported within the spinal column S to stabilize the vertebrae V to allow the fusion of fractured vertebrae as well as to allow healing of damaged or ruptured discs D that may be present in the spinal column S. Instrumentation in the form of rods rigidly connected to the stems 26 of the installed pedicle screws 20 are joined together to form a rigid lattice-type network (not shown) to stabilize and fixate the components of the spine to prevent or at least minimize any further movement or damage and to promote healing. The instrumentation that provides this fixation and stabilization of the spine is well known and therefore will not be further discussed in this application.

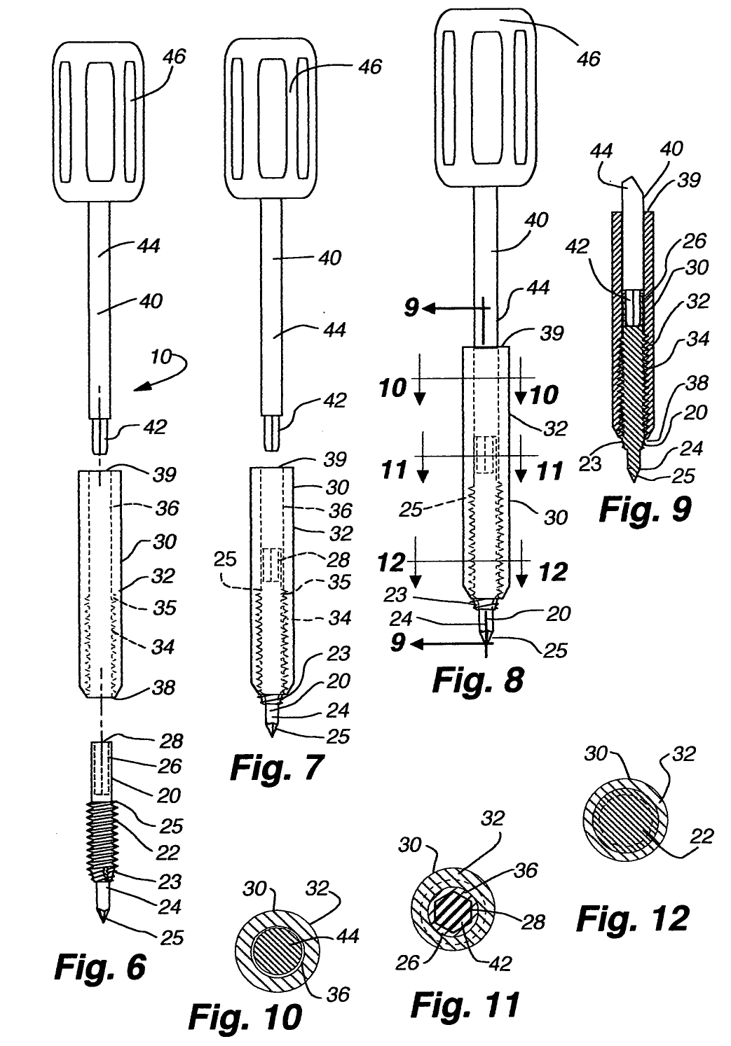

As seen in FIGS. 6-12 the pedicle screw and guide sleeve system 10 is shown in various views. The system 10 includes the pedicle screw 20, the guide support sleeve 30 and the driver 40. The pedicle screw includes a threaded central body portion 22, a distal guide end 24 along with a sharp pointed cutting end 25 and a proximal, smooth cylindrical barrel or stem 26. A suitably configured socket or drive recess 28 is provided in the stem 26 for receiving and coupling with the drive tip 42 of the driver 40. The guide support sleeve 30 includes the hollow cylindrical body 32 which includes an internal threaded portion 34 which matches the threads and the length or at least a partial length of the threaded body 22 of the pedicle screw 20. Varying lengths of sleeve 30 can be manufactured to fit different depths of soft tissues encountered in patients. The balance of the cavity in the guide support sleeve has an internal diameter at least large enough to slidably receive the shank 44 of the driver 40.

The pedicle screw 20 has a relatively narrow diameter cross section with respect to its overall length. The small diameter of the pedicle screw 20 which is approximately 5-8 mm is desirable from the standpoint that the screw can provide adequate seating within the bone structure for the purpose of anchoring the instrumentation without producing excessive lateral stress on the bone during the thread cutting process which could cause the bone to split or fracture inflicting additional trauma and injury on the patient. Various diameters of the threshold screw are manufactured to accommodate different patient sizes.

As seen in FIG. 6 the distal guide end 24 of the pedicle screw 20 is approximately 10-15 millimeters in length while the threaded body portion 22 is approximately 25-60 millimeters in length to accommodate varying patient size requirements. The smooth stem portion 26 also has a length of approximately 10-20 millimeters. Thus the guide end 24 and the stem end 26 have a ratio of approximately 1:4 and 1:2, respectively, with respect to the threaded body portion 22 of the pedicle screw 20. These dimensions can vary depending upon the overall size of the patient. The outer end 25 of the guide 24 converges into a sharp point and has a plurality of sharp edges making up the point. Thus, as the pedicle screw is rotated the sharp edges cause a boring or reaming effect which opens up an aperture or bore in the bone to allow introduction of the cutting threads 23 of the threaded body portion 22.

The cutting threads 23 are of the self tapping configuration which are well known in the art and provide a thread cutting function in the bone mass as the pedicle screw is rotated. In most cases the configuration of the threads in the body portion 22 and the cutting threads 23 will be of the right hand configuration so that the cutting function will take place as the screw is turned clockwise as viewed from its proximal end 26.

As will be discussed later, it is also possible to provide one or more longitudinal flutes or slots 125 along at least part or the entire length of the threaded body portion 22 of the pedicle screw 20 in order to allow the bone chips and debris that are produced during the thread cutting process to be moved longitudinally backward along the screw so as to provide relief for the removal of the debris.

In preparation for use, the pedicle screw 20 is reverse threaded into the end of the internal threaded bore of the guide support sleeve 30 so that the uppermost or proximal threads of the pedicle screw 20 will engage the innermost internal threads of the support sleeve 30. Since the length of the internal threads in the sleeve match the screw threads, the screw threads are concealed within the sleeve during placement and installation. Thus, the pedicle screw 20 is firmly seated, housed and supported within the support sleeve 30. The driver 40 can be used to assemble the pedicle screw within the sleeve 30 by insertion of the shank 44 into the interior cavity 36 of the support sleeve 30. In this way the engagement or drive end 42 of the driver 40 is inserted into the recess 28 provided in the proximal end of the pedicle screw 20 and the screw 20 is then turned backwards to thread it into the sleeve. The system 10 is properly assembled when the pedicle screw 20 is fully inserted within the support sleeve 30 and the driver 40 is inserted to engage the pedicle screw. In this configuration the pedicle screw and guide support assembly 10 is ready for percutaneous installation into the desired location within a vertebrae.

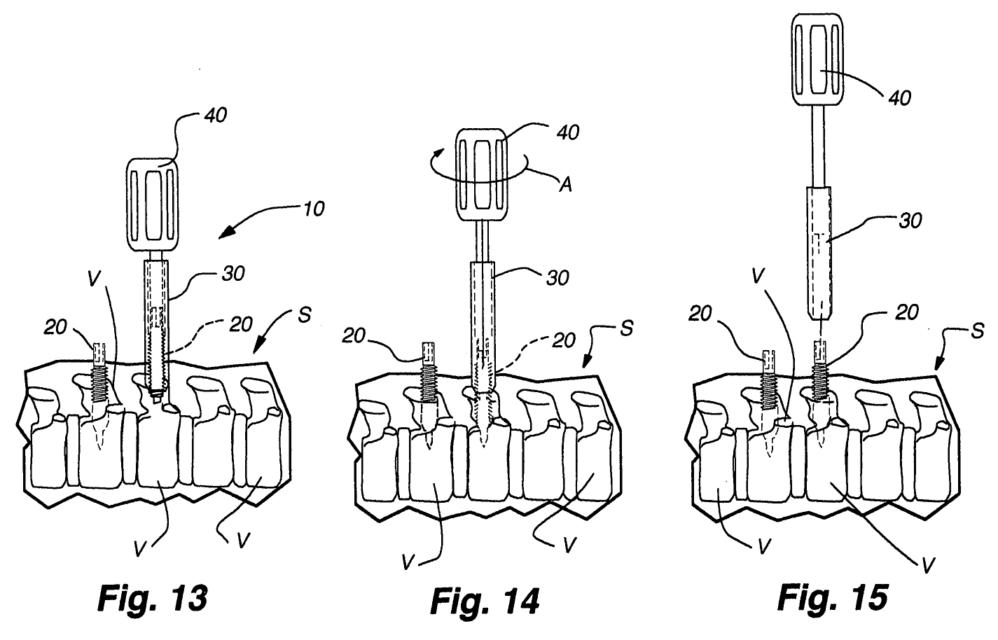

FIGS. 13-15 shows the actual use of the assembled components or system 10 for installation of the pedicle screw 20 into the bone mass. The guide end 24 of the pedicle screw 20 assembled within the guide support sleeve 30 along with the driver 40 is positioned in contact with the bone mass or vertebrae through a small incision made through the skin and soft tissue of the patient. Once the pedicle screw is properly positioned, the sharp point 25 of the distal end 24 of the screw can be set in the surface of the bone by a light tap on the handle of the driver 40 either by the hand of the surgeon or by a small light weight instrument.

Once the pedicle screw 20 is properly positioned and set and the system 10 is aligned with the respective vertebrae, the driver 40 is then rotated in a clockwise direction as shown by arrow A along with the application of longitudinal force on the driver 40. This action rotates the guide 24 with the cutting point 25 causing the reaming of an opening or aperture in the pedicle area of the vertebrae V. This continuous rotation of the pedicle screw 20, support sleeve 30 and driver 40 quickly generates an aperture through the dense outer layer of the bone structure and into the softer inner nucleus of the vertebrae. The continuous rotation of the driver 40 causes the cutting threads 23 of the screw 20 and the distal edge of the sleeve 30 to engage the surface of the bone and the screw 20 to cut new additional threads into the aperture reamed by the guide end 24. The support sleeve 30 stops rotating upon contacting the bone and is then held rigid and new threads are cut through the outer dense layer of the bone structure as the screw emerges from the sleeve.

The continued rotation of the driver 40 causes the screw 20 to extend further outward from the support sleeve 30 guiding the screw 20 into the newly formed aperture in the vertebrae. The screw 20 can be threaded entirely into the vertebrae or if desired can be turned leaving a predetermined portion of the threaded body 22 of the screw 20 exposed above the surface of the vertebrae V. When the threads are partially exposed it is then necessary to turn the support sleeve backwards so as to withdraw the support sleeve from the uppermost part of the screw. Once this has been accomplished the support sleeve 30 and driver 40 are then easily retracted leaving the pedicle screw 20 firmly anchored within the bone structure of the vertebrae. The use of the support sleeve 30 during the rotational insertion of the pedicle screw 20 into the vertebrae keeps the threads of the screw from coming in contact with this tissue during installation which can cause extensive damage and irritation during the surgical procedure.

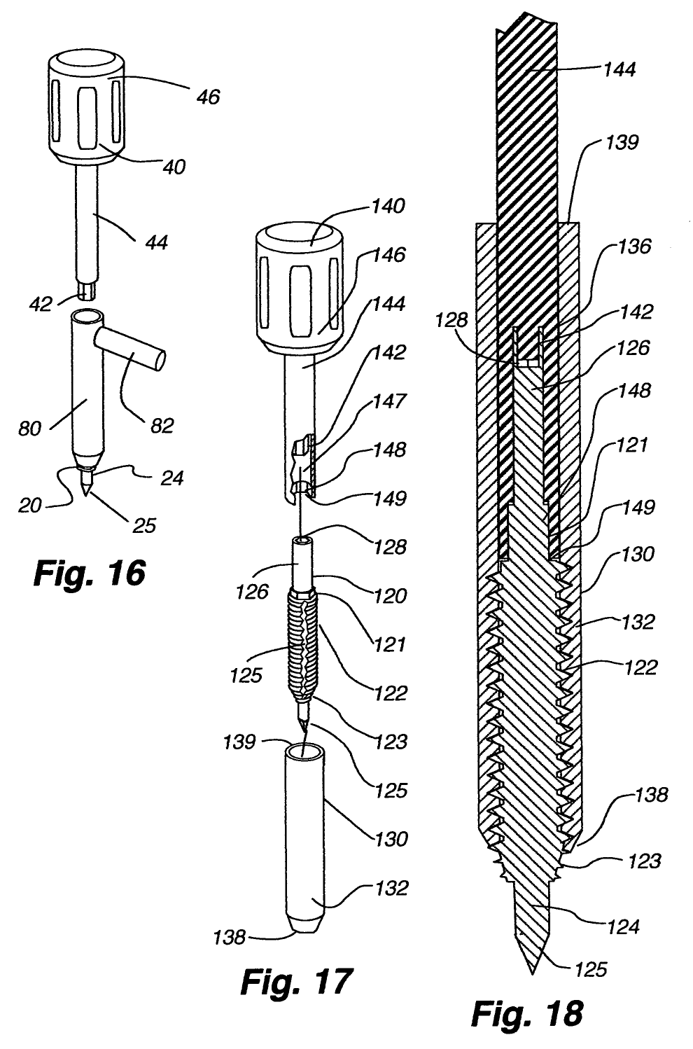

FIG. 16 shows another embodiment of the support sleeve 80 which includes all of the attributes of the previously described support sleeve 30 except that a handle or grip 82 is provided extending transversely from the upper end of the support sleeve 80. It is desirable that a suitable grip be provided on the support sleeve 80 in order to firmly hold the sleeve 80 during the installation of the pedicle screw 20. The handle 82 facilitates the alignment and positioning of the sleeve 80 even though the sleeve 80 may become difficult to secure due to fluids that may be present during the surgical procedure.

Another embodiment of the pedicle screw and guide support sleeve of the present invention is shown in FIG. 17. In this embodiment, a double drive configuration is provided for the coupling between the pedicle screw 120 and the driver 140. The pedicle screw 120 includes the threaded body 122, smooth cylindrical stem 126 and a drive socket recess 128. The driver shank 144 is slightly greater in diameter than the previous embodiments with the driver tip 142 recessed within a hollow cavity 147 at the end 149 of the driver shank 144. The inner surface of the drive cavity 147 includes a hexagonal socket 148 formed near the outer edge 149 of the shank 144. The area above the threads of the pedicle screw 120 is formed into a hexagonal drive coupling 121 which forms the transition between the threaded body portion 122 and the stem 126. The internal cavity 147 formed in the outer end of the driver shank 144 has a diameter large enough to pass over the outer surface of the stem 126. Thus, to make the drive connection between the driver 140 and the pedicle screw 120, the driver shank 144 is inserted over the stem 126. The distance between the drive tip 122 and the hexagonal socket 148 is arranged to coincide with the distance between the hexagonal drive coupling 121 and the receptacle 128 on the pedicle screw 120. It is to be understood that even though a double drive connection is provided only one or the other of the drive tip 142 or the hexagonal socket 148 may be necessary depending upon the strength of materials utilized in forming the driver shank 144 and the pedicle screw 120. This decision can be made and based not only on the materials that are used in the fabrication of these components but also on the type of orthopedic surgery that is anticipated as well as the bone mass that is to be encountered.

It is also to be noted in FIG. 17 that a longitudinal flute 125 can be provided either partially or the full length of the threaded body portion 122 of the pedicle screw 120 in order to provide relief for the removal of the bone chips or debris that is produced during the thread cutting process when the pedicle screw 120 is installed into the vertebrae or other bone mass. This debris is moved into the support sleeve 30 where it can be removed along with the sleeve. It has been found beneficial to provide relief for this debris which then allows the bone chips and debris that are formed during the threading process to be moved away from the cutting threads so that the cutting threads will not be clogged or bound by the bone material.

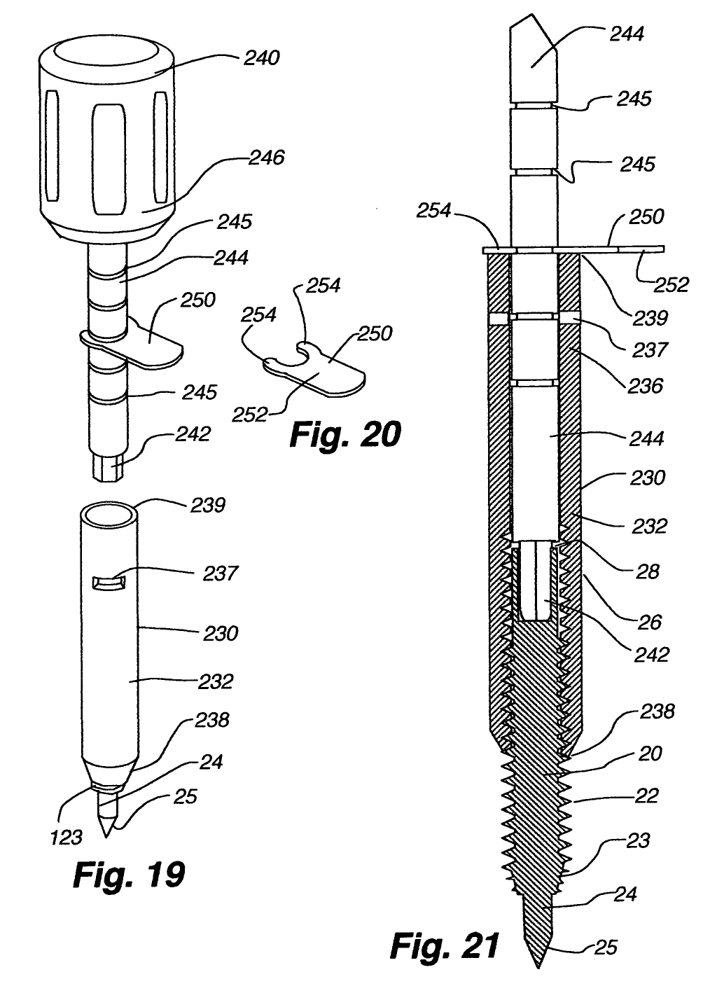

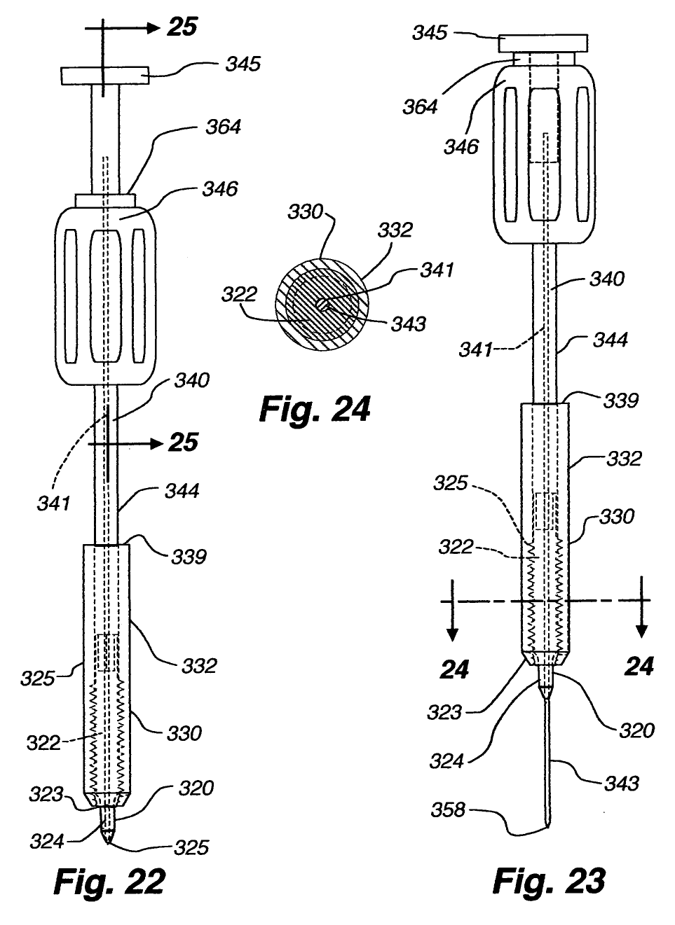

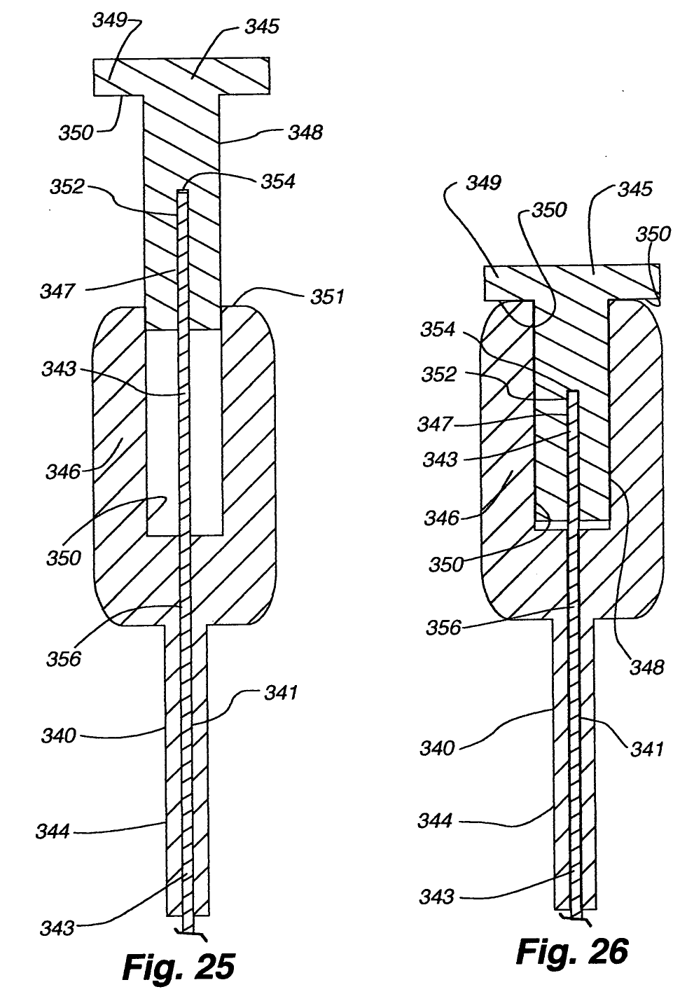

FIGS. 19-21 show another embodiment of the present invention in which lines or indices are spacedly scribed along the shank of the driver 240. In some cases these marks can be peripheral slots 245 provided around the shank 244 of the driver 240 which are sized to receive a clip retainer or locking ring 250.

The locking ring 250 includes a handle portion 252 and outer bifurcated ends 254. The outer ends 254 circumscribe a partial circle and converge slightly towards each other so that the ends can pass around the circumference or slots 245 formed in the shank 244 of the driver 240. In this way the locking ring 250 can be slidably inserted and held in any one of the slotted grooves 245.

Once the pedicle screw 20 is inserted into the supportive sleeve 230, the driver 240 can be inserted into the sleeve 230 to mate with the upper end of the pedicle screw 220. When the driver is positioned the locking ring can be inserted into the correct circumferential slot 245 which has a distance from the upper edge 239 of the support sleeve 230 to match the length of the threads on the pedicle screw 20. The driver 240 is then rotated until the locking ring 250 contacts the upper edge 239 of the support sleeve 230. At this point the pedicle screw 20 has been installed a predetermined distance into the vertebrae V. In this way, the pedicle screw can not be driven too far into the vertebrae wherein the guide end 24 of the pedicle screw might penetrate and exit the vertebrae.

In addition, by strategically positioning a pair of slots 237 on each side of the support sleeve 230 a locking ring 250 can be inserted around the outer circumference of the sleeve 230 to properly engage one of the peripheral slots 245 formed in the shank 244 of the driver 240. In this way the driver can be locked within the support sleeve 230 which secures the three components together in their properly assembled position. In this way the pedicle screw and support sleeve system is rigidly held together in preparation for the surgical installation of the pedicle screw. Once the system has been properly positioned subcutaneously, the locking ring 250 is removed from the outer surface of the support sleeve 230 allowing the driver 240 to be freely rotated for the installation of the screw. Prior to the threading process, the locking ring 250 can be repositioned in one of the exposed peripheral slots on the shank 244 at the proper distance to limit the length of the threaded portion of the pedicle screw that is to be inserted within the vertebrae.

It is to be understood that the spacing between the peripheral slots 245 is expected to be relatively equal in units that are anticipated to be required for insertion of various lengths of the pedicle screws that are to be used. This is also determined by the overall length of threaded body portion of the pedicle screw which can vary depending upon the overall size and weight of the patient. The dimensions of the pedicle screw 20 can also be varied such as by lengthening or shortening of the guide end 24 as well as the stem end 26. In addition, the number, pitch and type of threads in the body portion 22 can be varied depending upon the anticipated type or condition of the bone, various bone densities and the required depth of the installed pedicle screw. In conjunction with the length of the threaded portion 22 the overall diameter of the screw in these various areas can also be adjusted either larger or smaller depending upon the anticipated size of the bone configuration within the spinal column S. It is anticipated that the overall number and size of the various pedicle screws 22, support sleeves 30 and corresponding driver 40 can be optimized to a reasonable number of combinations to cover a wide range of patient sizes that are normally anticipated.

One of the problems that has been encountered in the past in the installation and insertion of orthopedic screw type threaded fasteners has been the guidance and verification of the fastener as it is being inserted into the bone mass. The difficulty here is to verify and be certain that the screw has not inadvertently exited the bone mass which in turn could cause traumatic damage to the internal organs of the body and in some cases catastrophic results. In order to accomplish this several types of image guiding systems can be employed to verify the insertion. One is the use of fluoroscopic x-ray equipment to provide continuous x-ray observations of the bone mass as the orthopedic screw is inserted. One of the major problems that occurs here is the fact that the x-ray projection is two dimensional and provides no depth perception with respect to the location of the screw and also provides extensive x-ray exposure to the patient as well as the surgeon. Another is a computer display generated by computer axial tomography, commonly called a “CAT scan” which provides a three dimensional view of the bone mass where the orthopedic screw is being installed. These image guidance systems are not continuous and instantaneous and only provide a spaced series of displays which are not real-time but are actually delayed exposures during the insertion process. Thus, the present state of the art does not provide a completely adequate guidance system to provide absolute confidence that the screw will not exit the surface of the bone mass.

In order to overcome this situation the present invention includes a manual locator device which is used prior to the actual insertion of the screw by providing an accurate projection of the screw as to depth and location prior to the actual insertion of the screw. In this way the position and alignment of the pedicle screw 322 and installation sleeve 330 can be verified prior to the actual implementation. The position of the locator rod can be accurately determined by the existing image guidance systems. In this way it can be easily determined that the screw is properly aligned and has sufficient clearance to not exit the surface of the bone mass.

To accomplish this a very thin cylindrical channel or passageway is formed completely through the screw installation system which means that the channel or passageway follows the longitudinal axis through the center of the driver handle 346, shank 340 and pedicle screw 322. The channel 341 exits through the distal guide end tip 325 and provides a clear passageway through the entire assembly for the locator rod 343. An installation cap 345 has a body 347 which is sized to slidably fit within the cavity 350 provided in the outer end of the handle 346 for the driver 340. A centrally positioned hole 354 can be provided within the body 347 of the cap 345. The outer end of the cap 345 includes an enlarged end portion 349 which has a flat under surface 350. The location rod 343 extends upwardly so that the end of the rod engages the cap 354 in the central hole 347. The locator rod 343 and cap 345 can be left as two separate parts or the rod can be attached to the cap within the hole 347 through the use of any attachment arrangement desired, such as a suitable adhesive. The actual length of the rod 343 and cap 345 is critical to the desired function of the device. The distal end of the rod 343 includes a sharpened point so that it can easily penetrate the bone mass upon insertion.

The length of the rod assembly is determined by measurement of the outer end of the distal guide end 325 of the screw 322, along the longitudinal axis of the screw and the driver 340 to the upper end of the hole 352 formed in the rod installation cap 345. For this measurement the installation cap 345 is precisely positioned within the cavity 350. The clearance distance between the top surface 351 of the drive handle 346 and the bottom surface 350 of the cap 345 is designed to precisely equal the length of the threaded portion of the pedicle screw 322. With the cap 345 held precisely in this position the rod is then measured to contact the upper end 354 of the hole 352 and then is cut precisely to this length. Thus, the end of the locator rod 343 is aligned with the end of the guide tip 325 as the proximal guide tip 320 is rotated and inserted into the outer surface of the bone mass until the end 323 of the installation sleeve 330 is in contact with the surface of the bone mass. With the pedicle screw installation assembly properly aligned the locator cap 345 is gently tapped by a suitable instrument to force the locator rod 343 into the bone mass until the lower end of the cap 350 contacts the top 351 of the driver handle 346.

With the locator rod 343 is positioned within the bone mass an image guidance system is then employed to accurately determine the precise location of the tip 358 of the locator rod 343. This accurately projects the precise location of the guidance tip 325 of the pedicle screw 322 after it has been inserted into the bone mass. The locator rod 343 is then withdrawn after the projected location for the screw has been determined.

It is also possible to provide additional adjustments to the actual depth and travel of the rod and cap by providing spacer disks 364. The disks 364 can have a central aperture which fits the outer diameter 348 of the body 347 of the cap 345 which allows the cap 345 to slide longitudinally with respect to the handle of the driver 340. The actual thickness of the disks 364 can be precisely determined to correspond with any number of threads of the pedicle screw 322 so as to actually limit the depth of the guide rod 343, if it is predetermined that only a certain number of threads will be inserted into the bone mass. In this way the depth of the location rod can coincide with the anticipated actual depth of the installed screw. It is understood that a number of disks 364 can be used for this purpose to adjust the final depth of the locator rod during its insertion. It is also understood that the disks can be sized to be inserted internally within the cavity 350 of the driver handle 346 and provide a similar function within the driver cavity 350 with respect to the movement of the cap 345.

It is important that the material which is used to fabricate the locator rod 343 must be compatible with bone and body tissue. The material must be extremely rigid and of high strength so that it will not bend or be deflected as it is forced into the bone mass. On the other hand it cannot be of a brittle nature which would allow it to possibly break off during insertion and leave it irretrievable embedded in the bone mass.

It is preferred that the components of the support sleeve and most especially the pedicle screw will be formed from a suitable rigid, non-corrosive material such as stainless steel or titanium or other materials such as synthetic resins, ceramics or rigid plastics. It is anticipated that any material can be used which meets the rigidity and strength requirements and is compatible with the patient's tissue. The handle 46 of the drive tool 40 can be of the ratcheting type which allows easier and more leveraged rotation of the drive tool during the surgical procedure. As an alternative an electric drive motor such as an electric drill can be attached to the screw assembly to provide the driving force.

The above disclosure sets forth a number of embodiments of the present invention described in detail with respect to the accompanying drawings. Those skilled in the art will appreciate that various changes, modifications, or other structural arrangements and other embodiments could be practiced under the teachings of the present invention without departing from the scope of this invention.

I claim:

1. An orthopedic bone attachment system for attaching objects to a bone mass, said system comprising:
(a) an elongated, cylindrical screw having a body, a distal end, and an opposite proximal end, said body including an outer surface having threads formed from the distal end and extending along a predetermined length of said outer surface of the body of the screw, the distal end of said screw having a short pointed guide probe for penetrating a bone mass and forming a bore for the body threads, the proximal end of said screw having an attachment extension for attaching objects and a driver coupling means, and
(b) a hollow, cylindrical support sleeve having a distal end and opposite proximal end, said sleeve including internal threads which correspond with the screw threads and which are formed from the distal end of the sleeve so that when the screw is inserted and enclosed within the sleeve a first thread of the screw is substantially even with the distal end of the sleeve, a central passageway is formed in the proximal end of the sleeve to receive the attachment extension of said screw.

2. An orthopedic bone attachment system as defined in claim 1 wherein first body threads in the distal end portion are self tapping threads so that threads can be cut into the bone mass for securing the screw.

3. An orthopedic bone attachment system as defined in claim 2 wherein the body threads extending from the distal end include one or more longitudinal flutes for carrying away debris from the thread cutting area.

4. An orthopedic bone attachment system as defined in claim 1 wherein the driver coupling means includes a recess which is compatible for receiving a driver device so that the screw can be rotated so that it will thread into said bone mass.

5. An orthopedic bone attachment system as defined in claim 4 wherein the coupling means includes a double receptacle formed in said extension whereby a driver device having a dual coupling configuration can mate with the double receptacle formed in the attachment extension on said screw so that the screw can be rotated.

6. An orthopedic bone attachment system as defined in claim 1 wherein said attachment extension on said pedicle screw is formed as a cylindrical surface.

7. An orthopedic bone attachment system as defined in claim 1 wherein the distal end of said support sleeve has a rounded edge surface whereby the support sleeve edge as it is inserted through an incision in the soft tissue of a patient will produce a minimally invasive penetration of the soft tissue and prevent contact of the screw and screw threads with the soft tissue.

8. An orthopedic bone attachment system as defined in claim 1 which further includes a driver device comprising an elongated shaft having an outer end containing a coupling device, said shaft being arranged to extend through the central passageway of the sleeve so that the coupling device can connect to the drive coupling means on the screw, and a grip portion at an opposite end of said shaft from said coupling device for turning the driver device and inserting said screw.

9. An orthopedic bone attachment system as defined in claim 8 wherein the driver coupling means on the screw and the coupling device on the driver device includes a plurality of configurations for simultaneously connecting the driver device and the screw for rotation and insertion of the screw.

10. An orthopedic bone attachment system as defined in claim 9 wherein the elongated shaft of said driver device is indexed with respect to the proximal end of said support sleeve whereby the threaded depth of the screw can be determined as the driver device is rotated for inserting the screw.

11. An orthopedic bone attachment system as defined in claim 10 wherein the shaft indexing is formed by a plurality of circumferential grooves and a locking clip is provided which fits a selected indexing groove to contact the proximal end of the sleeve to limit the insertion depth of the screw with respect to the distal end of the support sleeve.

12. An orthopedic bone attachment system as defined in claim 1 wherein the threads formed in the outer surface of the screw body are continuous.

13. An orthopedic bone attachment system as defined in claim 1 wherein the short pointed guide probe on the distal end of said screw has a diameter which has a ratio of approximately 1:2 to the diameter of the cylindrical screw.

14. An orthopedic bone attachment system as defined in claim 1 wherein the support sleeve includes a protrusion which is a grip for holding and aligning the support sleeve while rotating and installing the screw.

15. An orthopedic bone attachment system for inserting a screw into a bone mass, said system comprising the combination of:
(a) an elongated screw having threads along a body portion, a short narrow pointed guide probe at a distal end of the screw and a cylindrical attachment extension at a proximal end, the proximal end of said screw having a drive attachment means,
(b) a hollow elongated cylindrical support sleeve having internal threads extending inwardly from a first end, said threads being arranged to correspond with the threads of said screw whereby the screw can be installed into said sleeve so as to be enclosed and supported within said sleeve, and
(c) a drive device having an elongated shaft which can pass through the sleeve and attach to the drive attachment means of said screw, a drive coupling is formed at the end of the shaft for connecting to the screw attachment means for rotating the screw so that the screw can be threadedly inserted into the bone mass while it is aligned and supported by said sleeve.

16. A bone attachment system as described in claim 15 wherein a central longitudinal passageway is formed in the elongated screw and connected drive device, a locator rod sized to slidable fit within said longitudinal passageway includes a sharp point at a distal end and a cap having a stop surface for contacting the drive device at a proximal end, the length of the locator rod between the distal end and the cap stop surface equals the length of the connected screw and drive device and the length of the threads that will enter the bone mass when the screw is inserted so that the screw can be positioned and aligned with respect to the bone mass and the locator rod driven into the bone mass until the cap stop surface contacts the drive device whereby the locator rod location can be determined by a suitable imaging device to verify that the screw will be properly located within the bone mass prior to its being inserted.

17. A method for inserting a minimally invasive orthopedic screw into a bone mass for attaching therapeutic instrumentation to said bone mass, said method including the steps of:
(a) forming an elongated orthopedic screw having a threaded body portion, a smaller pointed guide probe distal end and a cylindrical attachment device formed at a proximal end of said screw, self tapping threads being formed as the first threads of said body threads in an area adjacent to the distal end of said screw,
(b) forming a thin hollow elongated cylindrical support sleeve having internal threads extending inwardly from a distal end which are sized to receive the threads of said orthopedic screw, threading the screw into said support sleeve whereby said threaded portion of the orthopedic screw is enclosed within said support sleeve,
(c) driving the guide probe on said screw into said bone mass until the distal end of said support sleeve and the distal end of the threaded portion of said screw are in contact with the surface of the bone mass,
(d) rotating the orthopedic screw while holding the support sleeve so that the screw will be threaded into the bone mass a predetermined distance, and removing the support sleeve so as to expose the cylindrical attachment device of said orthopedic screw whereby therapeutic instrumentation can be attached to the screw and thus to the bone mass.

18. A method for inserting a minimally invasive orthopedic screw as described in claim 17 which further includes the rigid alignment of the support sleeve at the same time that the screw is rotated to verify the proper position of the screw during the time that the screw is installed into the bone mass.

19. A locator for an orthopedic screw for predicting the final location of the screw in a bone mass prior to the insertion of the screw, the locator comprising:
(a) a threaded orthopedic screw for insertion in a bone mass, said screw having a longitudinal axis and a guide end for aiding the threads in penetrating the bone mass,
(b) an elongated drive device for connecting to the screw so that the screw can be turned and threaded into the bone mass, said drive device having an elongated axis which is aligned with the axis of the screw,
(c) said screw and drive device having a small diameter passageway extending through the screw and drive device and formed concentric with the longitudinal axis, and
(d) an elongated thin rod having a point formed at one end and a cap formed at the opposite end, said rod being sized to slidably fit within the longitudinal passageway of said screw and drive device, said cap having a stop surface that contacts said drive device when the rod is slidably positioned within the passageway, the length of the rod being predetermined to equal the combined length of the screw and drive device and the intended thread depth of the screw upon insertion, said rod being arranged so that when the screw and drive device are properly aligned with the bone mass the rod can be driven into the bone mass until the rod cap contacts the drive device whereby the location of the rod within the bone mass can be observed by an image guidance system to accurately project the final position of the installed screw.

20. The locator for an orthopedic screw as defined in claim 19 wherein one or more spacers disks having a predetermined thickness can be inserted over the rod adjacent the cap end whereby the spacer will limit the travel of the rod into the bone mass to a predetermined depth when only partial insertion of the screw threads are intended.

21. A method for projecting the location of an orthopedic screw in a bone mass prior to installing the screw, the method comprising the steps of:
(a) assembling an elongated orthopedic screw and an elongated drive device to be drivingly connected to said screw for threading the screw into the bone mass, both of the screw and drive device having an aligned longitudinal axis, said screw having a plurality of threads extending from a distal end along a body portion;
(b) providing a narrow passageway completely through the screw and drive device when assembled, the passageway being coaxial with the longitudinal axis of the assembly;
(c) forming a thin elongated locator rod having a diameter which will slidably fit said coaxial passageway, said rod having an enlarged stop at one end and has a predetermined length which equals the overall assembled length of the screw and drive device plus the length of the screw threads to be installed in said bone mass;
(d) positioning the assembled screw and drive device on the desired area of the bone mass in a properly aligned position for the screw;
(e) driving the locator rod into the bone mass until the enlarged stop contacts the drive device;
(f) determining the location of the rod in the bone mass and verifying that it correctly projects the position of the screw when installed; and
(g) withdraw rod and install the screw.
